(19) United States
(12) Plant Patent Application Publication
(10) Pub. No.: US 2013/0291238 P1
(43) Pub. Date: Oct. 31, 2013

(54) Lilac tree named 'Summer Storm'

(50) ;

(71) Applicants: Joshua Ryan Robinson, McMinnville, OR (US); Rock Kelly, Northfield, MN (US)

(72) Inventors: Joshua Ryan Robinson, McMinnville, OR (US); Rock Kelly, Northfield, MN (US)

(73) Assignees: ROBINSON NURSERY Inc.; BACHMAN'S Inc.

(21) Appl. No.: 13/506,563

(22) Filed: Apr. 30, 2012

(51) Int. Cl. A01H 5/00

(52) U.S. Cl. PLT/248

(57) ABSTRACT

A new seedling selection of Syringa reticulata named ‘Summer Storm’ characterized by its consistency, vigorous growth and hardiness.

The present invention comprises a new and distinct cultivar of a seedling selection of a Japanese tree lilac, botanically known as Syringa reticulata

The parent tree is growing in a cultivated area on the home grounds of co-inventor Rock Kelly at Bachman's Nursery, 6877 235^{th} St. W. Farmington Minn. 55024. six miles southwest of Farmington city center, in Dakota County, It is in the township of Eureka 10, in range 20 W. The new cultivar is a result of a Syringa reticulata planting where ‘Summer Storm’ stood out from a planting of 200 other reticulata seedlings. The difference was very noticeable as it was growing twice as fast as the other seedlings and maintaining a central leader. The trees were bought in from co-inventor Robinson Nursery as a two year plant in 1994. Bachman Nurseries production manager Rock Kelly noticed how vigorous it was, selected it and replanted it in a cultivated area at Bachman's Nursery. The Syringa reticulata selection ‘Summer Storm’ proved to be worthy of cultivar status.

The ‘Summer Storm’ cultivar is broadly upright in growth habit. It is wider at the bottom and gets more fastigiate as it puts on height. It is proven Hardy to zone 3. ‘Summer Storm’ withstood a winter of negative 35 degrees with no damage.

Cultivar ‘Summer Storm’ has been successfully asexually propagated at co-inventor Robinson Nursery, by collecting wood from the parent tree and budding or grafting onto Syringa reticulata. The success rate is at about 95% with no incompatibility between the scion and rootstock.

Buds set on a two year root stocks have produced six to nine feet of growth in one growing season. Asexually propagated trees of the ‘Summer Storm’ cultivar have maintained the unique characteristics which distinguish this cultivar selection from its parent plant Syringa reticulata.

The ‘Summer Storm’ tree lilac at 20 years of age is twenty feet tall and 12 feet wide. The Broadly upright structure will have several uses in the landscape. It can be planted on city streets where limited space is available. It could also be used a screen where there is a large amount of space available. It would lend itself well as a specimen tree for a house, golf course, or architectural design for box planting.

The “Summer Storm” lilac exhibits remarkably consistent vigor. As a one year whip on a two year root stock it had a size range of 6′-8′ in comparison with the Syringa reticulata and the cultivars ‘Ivory silk’ and ‘Snow Dance’ which were graded out to be in the range of 3′-7′

### DETAILED BOTANICAL DESCRIPTION

In the following description, color references are made to The Royal Horticultural Society Colour Chart.

### BRIEF DESCRIPTION OF THE DRAWINGS

The accompanying colored photographs illustrate the appearance and color of the new “Summer Storm” lilac. These photographs show the color as accurately as is reasonably possible to obtain on colored reproduction of this type. Actual foliage colors may differ slightly due to light reflections.

FIG. 1 depicts the initially discovered tree named “Summer Storm” holding summer foliage and broadly narrow form.

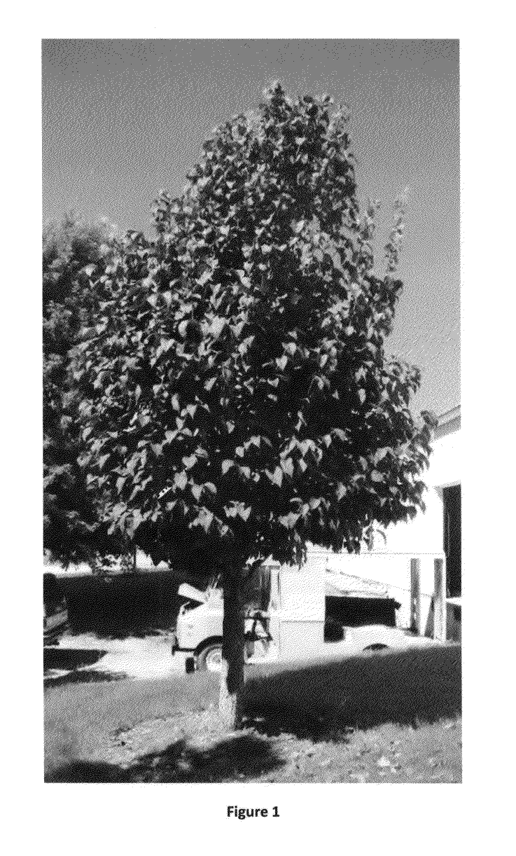

FIG. 2 depicts the ‘Summer Storm’ tree with no foliage

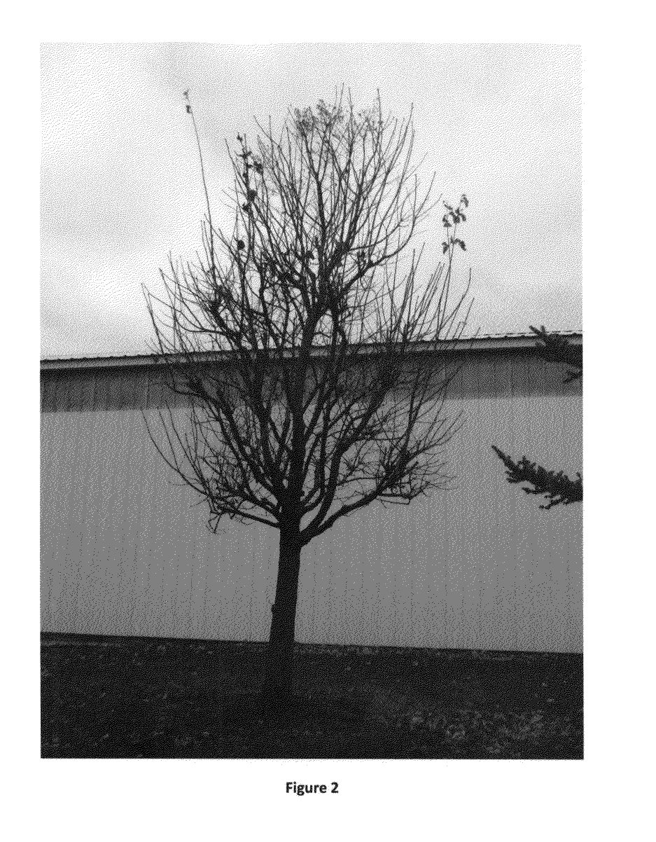

FIG. 3 depicts the first asexually propagated (chip budded) tree of the cultivar ‘Summer Storm’

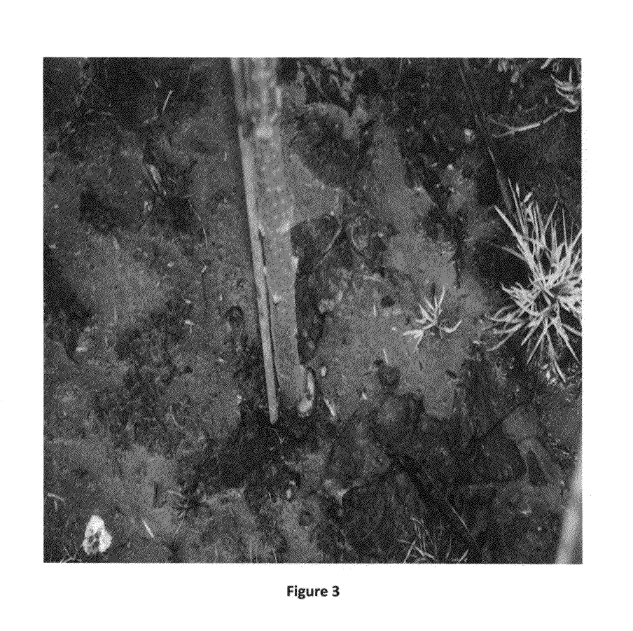

FIG. 4 depicts the foliage of the ‘Summer Storm’ cultivar both upper and lower surface of the leaf.

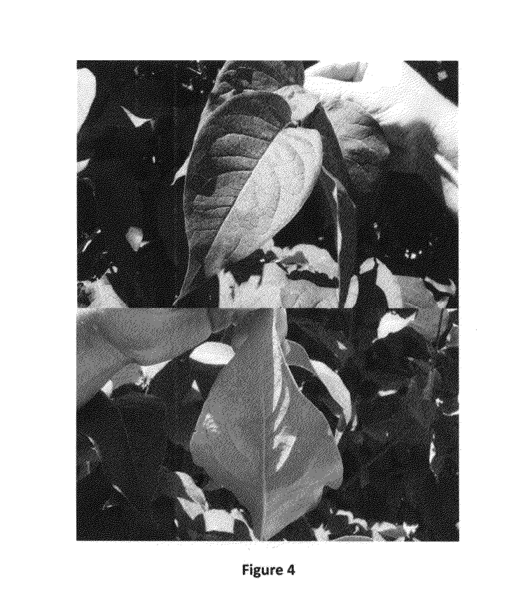

FIG. 5 Depicts the fruit of the ‘Summer Storm.

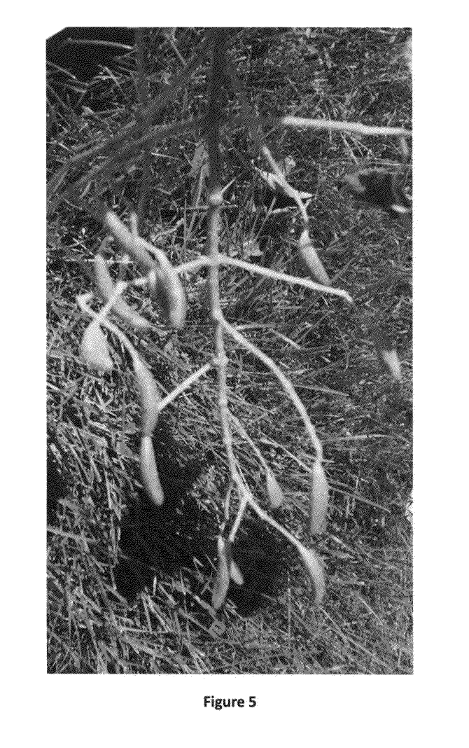

FIG. 6 Depicts the stem, including axillary buds.

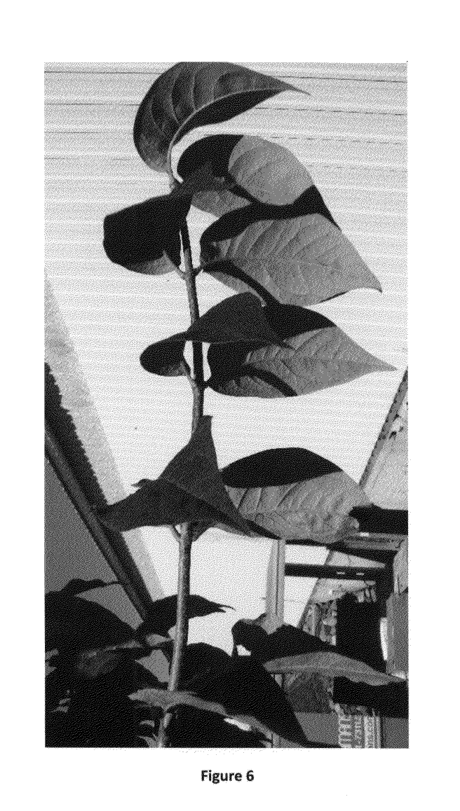

### THE PLANT

Tree, Syringa reticulata selection ‘Summer Storm’
Name: Syringa reticulata ‘Summer Storm’
Parentage: Seedling selection, 1994
Classification: Syringa reticulata
Where discovered: The selection was discovered at Bachman's Nursery Inc. 6877 235^{th} St. W. Farmington Minn., 55024 Dakota County, township of Eureka 10, in range 20 W. This plant has been has been asexually reproduced by grafting (chip budding) onto Syringa reticulata root stock. Presently propagation is being done in McMinnville, Oreg. by one wholesale nursery, co-inventor Robinson Nursery.
   Form.—Tree or multi stem.
   Shape.—Excurrent, broadly upright.
   Height.—5.5 m (18′) Spread 3 m (10′).
   Trunk size.—(dbh) (diameter of trunk at 135 cm above ground level) : 17.1 cm.
   Base.—cylindrical.
   Growth rate.—Fast.
   Strength.—Excellent.
   Age.—20 years from seedling.
   Bark.—(trunk) brown to light grey brown (N199 B) slight exfoliation giving way to yellow green (N144 A) (course in texture; small linear blocks, size 0.25-1 cm (165 D).
   Branches.—Angle of attachment: most branches between 45 and 55 degrees with a range of 40 to 60 degrees.
   Spacing.—Close. Small twig bark (<0.75 cm diameter) (Growing season) greenish, smooth (152 A & B) (Dormant season) red-brown, rough (200 B).
   Lenticels.—small (average=0.75 cm) scattered, linear, raised, color (165 D)
Leaves:
   Petiole.—Length: 2-3 cm average=1.5 cm Color: yellow-green (152 D) Texture: bumpy Lamina: Length: (12.7-17.15 cm, average=14.9 cm) Width: 4.5″-2.5″ (6.3-11.4 cm, average=8.9 cm) Form: broadly-ovate to ovate, acuminate, rounded or subcordate, Glabrous. Margin: smooth, entire. Texture: Leathery, flat, smooth, glabrous above and reticulate beneath. Quantity: Moderate Color: (summer) Upper side: dark green (135A & 136A) (autumnal) color changes to light green (144A) Lower side: young and old leaves are light-green (139D) Ribs and Veins: 7-9 pairs of veins (greenish yellow (145C).
Buds: Vegetative Large (0.5 cm-1 cm average=0.75cm) sessile, subglobose, 3-4 sets of scales the color is brown (200D)
Flowers: Perfect, Size: large, panicle form (length 6-12 inches average=9 inches) (width 6-10 inches average=8 inches) creamy white (NN 155A)
Fruit: scimitar-shaped, long capsule Size :(0.75-1 inch average=0.85 inch) Dehiscent
   Color.—of fruit green (141D)
   Peduncle length.—(1.25-1.75 inches average=1.5 inches)
   Peduncle color.—yellow (150C)
Has this plant ever been offered for sale: NO
Compare this new variety: with others of the same species and with its parent or parents; state the distinguishing characteristics of the new cultivar and its advantages. The selection was chosen because it is consistent and extremely vigorous in comparison to the other species available. It has been documented that compared to Syringa reticulata it has grown twice as fast in the 1^{st} and 2^{nd} year in production at Robinson Nursery, and the 3^{rd}, 4^{th}, and 5^{th} year in production at Bachman's Nursery. In addition to its vigor it is also very consistent compared to the seedlings and other cultivars. At Robinson Nursery there are plantings of the cultivars ‘Ivory Silk’ ‘Snow Dance’ and the seedling Syringa reticulata. The variance in ‘Summer Storm’ is consistently within 3 feet of one another 6-8 feet while the other cultivars range from 3-7 feet. The percentage of ‘Summer Storm’ to reach the height of 7′ or taller was 80% where all other species and the seedling were 30%.The combination of the vigor and consistency that it grows when lined out will be a big improvement compared to what is currently available today. ‘Summer Storm’ was graded as follows: 20% were 6′ 60% were 7′ and 20% were 8′. This is remarkable as with ‘Ivory Silk’ 15% were 3′ 20% were 4′ 20% were 5′ 30% were 6′ and 15% were 7′. Our grading records dating back 20 years show similar percentages for ‘Ivory Silk.’ When it comes to dollars this will make the tree more profitable as the growers can estimate what to expect, but with other varieties of Syringa reticulata you can't. With the consistent vigor comes predictability which is a great asset to tree growers.

It is claimed:

1. A new and distinct cultivar of Syringa reticulata named ‘Summer Storm’, as illustrated and described.
